(19) United States
(12) Patent Application Publication
(10) Pub. No.: US 2009/0275531 A1
(43) Pub. Date: Nov. 5, 2009

(54) Method for producing starch networks and initial products

(75) Inventors: Rolf Muller, Zurich (CH); Federico Innerebner, Zurich (CH)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: INNOGEL AG

(21) Appl. No.: 12/289,097

(22) Filed: Oct. 20, 2008

Related U.S. Application Data
(63) Continuation of application No. 10/515,644, filed on Jul. 11, 2005, now abandoned.

(30) Foreign Application Priority Data
Mar. 28, 2003 (DE) 103 14 418.8

(51) Int. Cl. A61K 31/715; C08B 31/00; C09D 103/04; A23L 1/0522

(52) U.S. Cl. 514/60; 536/47; 106/206.1; 426/661; 428/542.8

(57) ABSTRACT

The invention relates to a method for producing starch networks. The method may include preparing a basic starch and preparing a networking starch. The method may further include manufacturing an initial product including the basic starch and the networking starch, and processing the initial product to yield the starch networks, such that the starch networks are at least partially heterocrystallized.

The present invention relates to the production of starch networks by means of new methods and initial products for the simple production of starch networks and presents the advantageous properties of such networks.

### PRIOR ART

The production of new starch networks based on mixtures of basic starches (VS) and networking starches (NS) is described in the Patent Application WO 03/035026 A2. In this situation the NS and their preparation are primarily decisive for the advantageous properties of the starch networks obtained. As a result of the various parameters of the preparation, the potential of the NS to form networks, especially by heterocrystallisation with VS can be optimally converted into advantageous product properties. The NS are prepared separately from VS, the essential preparation steps including at least partially dissolving these starches as well as, if necessary, overheating or undercooling. In a next step the prepared NS is mixed with completely or partially plasticised VS so that a networking starch fluid (NSF), i.e., a networking solution or melt is obtained which under suitable conditions can subsequently be obtained as a network. After preparation of the NSF, this can be shaped into a shaped body, wherein or whereafter network formation is initiated, usually triggered by a reduction in the temperature and/or the softener content, especially the water content of the NSF. The method wherein VS and NS components are prepared separately (Split), mixed and then directly (Continuous) further processed to give the end product is known as the Split Continuous Process (SCP). Apart from the advantage of flexibility and the advantage that the NS can be optimally prepared for starch networks (for example, stabilisation of the NS solution, undercooling, production of nuclei for network formation under difficult conditions), this SCP method has the following disadvantages which make it difficult to implement rapidly on a broad industrial scale:

A. The separate processing of VS and NS is more expensive compared with a method where the components are prepared together (Together Continuous Process, TCP) and commonly used preparation methods must be suitably modified. Likewise, the separate processing of NS is more complicated and liable to breakdown compared with a TCP method, since significantly more process parameters need to be optimised and regulated. As a result, the widespread industrial implementation of the new technology to manufacture new types of starch networks is difficult. The simpler a new method is, the easier it is to implement it on a wide industrial scale.
B. Since the preparation of an NSF is directly linked to the further processing to form the end product (Continuous Process), every manufacturer of an end product must cope with all the preparation steps. In contrast thereto, a manufacturer of conventional plastic films, for example, need not concern himself with the preparation of specific polymer qualities or mixtures. He can buy in such already-prepared substances, for example, in the form of granules and concentrate merely on transforming these into the end product. Such a discontinuous sequence (Discontinuous Process, DP) is thus also desirable for the manufacture of products based on starch networks and will also make it significantly easier to implement the new technology industrially on a wide scale.

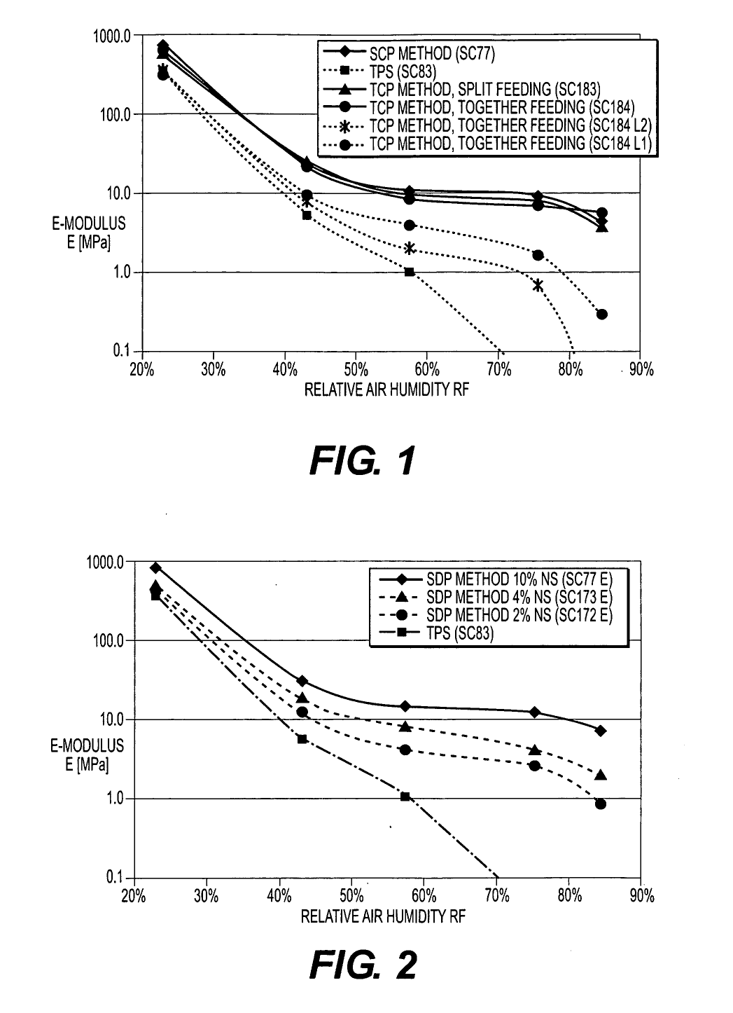

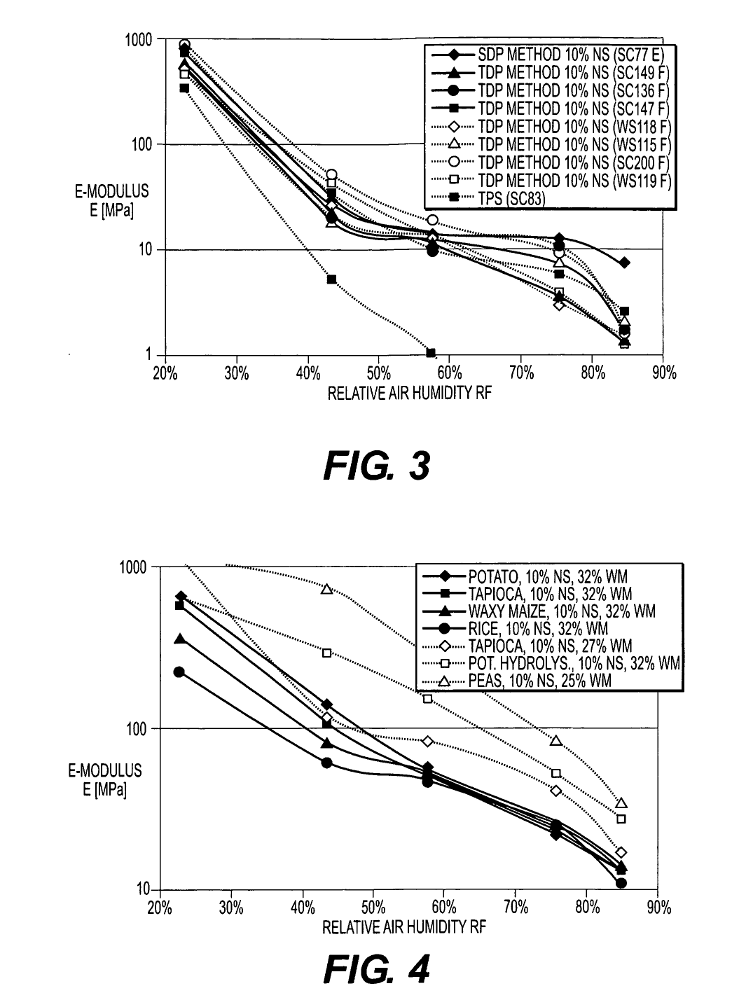

A method similar to the TCP method has already been described in the unexamined laid-open patent publication DE 198 52 826 A1 for the manufacture of thermoplastic polymer mixtures, wherein however no starch network was obtained. The poly-alpha-1,4-D-glucan (PG) used was in this case softened with glycerol, wherein the x-ray spectrum in FIG. 2 of said application clearly shows that this softened PG is present with very high crystallinity (this is a synthetically produced high-quality linear starch which crystallises very well and forms very stable crystallites). In this state, the PG was mixed into a melt of thermoplastic starch at temperatures around 160-180° C., wherein it is mentioned in the description and in claim 10 that the water content in the melt accounted for less than 5%. Under these conditions, the crystallites of the PG are distributed in the TPS melt but it is not possible for the PG molecules to dissolve completely in the TPS melt since appreciable fractions of water >25% are necessary for this and then only in the presence of strong shear forces. The analysis of graded mixtures of PG with TPS then also showed a clear reduction in the E modulus from 184 MPa to just 24.2 MPa with a 25% PG fraction (Table 1), a clear indication that the PG crystallites were embedded almost unchanged in the TPS matrix and the glycerol of the PG softened with glycerol diffused into the TPS matrix (crystalline PG can only bind small quantities of glycerol and this only on the surface). This is also confirmed in FIG. 1 from which it can be seen that the crystallinity of the mixture increases linearly with the PG fraction which is then to be expected if the PG is worked into the TPS matrix as filler, unchanged in its crystallinity. It is thus clear that in the method specified in said Application, no starch networks were obtained. In the starch networks which could be obtained by means of the TCP method in the present Application, a solution of networking starch (NS) in a TPS melt was obtained at water contents around 35% whereby a massive increase in the E modulus resulted even with a 10% NS fraction in the entire range of relative air humidity RF (FIG. 1 in this application). At this point, it may be mentioned that precisely the difficulties of dissolving NS, i.e., at least partly crystalline starch, resulted in the development of the SCP method where the NS is dissolved separately from the TPS with water contents of typically 50-95% and overheating techniques are applied for complete dissolution. In the TCP method however, it has now been possible to dissolve NS in a high-molecular starch melt at lower water contents of typically 25-50% in the presence of strong shear forces and to distribute this in a molecular-disperse fashion so that a networking starch fluid is formed from which starch networks can be obtained.

The problems of producing a solution of PG resulted in a method which is very similar to the SCP method. Unexamined laid-open patent publication DE 100 22 095 A1 describes starch gels which were obtained using PG and a further starch wherein a solution of PG is mixed with a solution of starch and precipitation could be successfully carried out with the formation of a gel (claim 9 of said application). Since the highly crystalline PG is considered to be insoluble in water (claim 2), the PG solution was obtained using stronger alkaline solutions (Example 1). After mixing these alkaline solutions containing dissolved PG with a starch solution, neutralisation was carried out using orthophosphoric acid, wherein the PG precipitated out and formed a gel together with the starch macromolecules. Potassium phosphate was also formed during the neutralisation. The concentration of the solutions of PG and starch in the various examples was 5% (Table 1, Table 2a, Table 2b) as well as 9 and 12% (Table 3), higher concentrations were not feasible by means of this method since even in 1 molar KOH alkaline solution a maximum of 12% PG could be dissolved and at this molarity, solution temperatures up to 25° C. could only be applied as a result of the degradation of the PG. Even with a concentration of 12% starch and 88% water, the gels obtained were extremely weak so that they could be damaged merely by touching. They also contained a fraction of glycerol which had been added to the starch solution in each case before the starch solution was mixed with the PG solution since the solubility of PG is reduced in the presence of glycerol (glycerol binds water whereby the quantity of water accessible for PG is reduced). In most examples, the glycerol fraction relative to the dry mixture was 50%. After the precipitation reaction, the thin gels were slowly dried by leaving to stand in the atmosphere whereby solid films having water contents around 5-10% were obtained, which could be analysed in the tensile test.

Against this background, the importance of the SCP method becomes clear, whereby, without alkaline solutions and their neutralisation by acids, molecularly disperse mixtures, i.e. networking starch fluids (NSF) and therefrom technically usable starch networks based primarily on heterocrystallisation could be obtained (wherein the crystallites, which form the network elements or the linking points of the networks, are formed from two or more types of starch molecules, especially from very small and readily crystallisable starch molecules and from large, normally non-crystallisable or barely crystallisable starch macromolecules) and this heterocrystallisation was possible at very much lower water contents than 88% (as far as water contents below 20%), as a result of which very much higher network densities can be adjusted, which result in the technical applications for the first time, among others in foodstuffs and pharmaceuticals (galenicals). The methods described in the present invention and further developed especially for widespread industrial application as well as the various initial products provide the basis for the widespread implementation of these new starch networks on an industrial scale.

### DESCRIPTION OF THE INVENTION

### The Together Process (TP) and the Discontinuous Process (DP)

The disadvantage of the SCP method specified under A could be overcome by the successful development of a Together Process (TP) whereby the separate preparation of VS and NS is unnecessary for many applications. Through suitable process management it could be achieved that the potential of NS to form networks, especially by heterocrystallisation with NS, can also be realised by joint preparation of VS and NS, i.e., the process wherein NS is preliminarily at least partly dissolved before mixing with VS, if necessary overheated and/or undercooled, could be eliminated. This was achieved on the one hand by specific process measures and on the other hand, by developing suitable formulations.

The essential process measure whereby a TP method can be made possible, relates to the joint conversion of VS and NS into an NSF at water contents in the range of 25-50% (process water, the fraction depends strongly on the type of NS), at high temperatures and especially at high shear rates (high speeds, high energy intake), whereby the potential of NS to form networks can be released and a separate preparation of a solution of NS is unnecessary. After an NSF has been obtained, in most cases some of the process water is removed again (retaining the NSF), for example, by evacuation techniques, whereby the temperature of the NSF can at the same time be lowered again. An important advantage of the separate preparation of NS and VS is that the solution of NS can be adjusted to a desired number of nuclei by overheating and/or undercooling. These nuclei are then important for adjusting high network densities after mixing with VS. In the TP method on the other hand, the number of nuclei can be adjusted by the NS still having residual structures during preparation of the NSF, which can act as nuclei. In addition, foreign nucleating agents can be added, as is also possible in the SCP method. With regard to the formulations suitable for a TP method it was found that especially suitable as NS for this purpose are NS types with a network-active chain length CLn,na in the range 7-300, preferably 10-100, more preferably 14-50, especially 16-30, most preferably 18-28. The crystallites of such NS are correspondingly small and can also be dissolved in the TP method. In the case of larger network-active chain lengths, higher water contents and higher temperatures must be used which can be problematical (thermal degradation) but for very short process times such NS can also still be processed. If the network-active chain length is too large, the crystallites are correspondingly large and stable. The CLn,na of amylose is reduced, for example, by branching which is why the almost completely linear PG of the unexamined laid-open patent publication DE 100 22 095 A1 presents particular problems. Natural amyloses or amyloses obtained from starches have significantly higher degrees of branching than the synthetically produced PG. In addition, the CLn,na is also reduced by substitutions such as hydroxypropylation, acetylation etc. which is why suitably modified amyloses are well suited. Amyloses having high CLn,na can also advantageously be used as NS if these are present predominantly in amorphous form, as is the case with many amyloses in the native starch grain, which facilitates the solution process. With regard to the kinetics of network formation, short chain amyloses are especially advantageous since they have a high mobility when dissolved in a starch melt and thus can rapidly form networks. Furthermore, in the TP method starches with a high molecular weight are advantageous as VS since they yield highly viscous melts which make it possible to transfer high shear forces to the NS.

The disadvantages of the SCP method specified under B could be overcome by developing a discontinuous method (Discontinuous Process). In this case, an initial product (VP) is manufactured in a first step, which product is capable of being stored and can be transported, and already contains the components VS and NS essential for starch networks, especially in a state favourable for the subsequent further processing to form the end product, for example, as frozen NSF (inhibited initial product, IVP).

The DP method can be executed both as an SDP method (Split Discontinuous Process) as also as a TDP method (Together Discontinuous Process). With regard to the separate preparation especially of the NS in the SDP method, reference is made to the SCP method described in Patent Application WO 03/035026 A2. The splitting of the preparation of NS and VS in the SDP method certainly results in a more complex process but also makes it possible to increase the scope of the process and has the advantage that the preparation of the starch mixture need not take place at the end consumer but can take place at a specialised manufacturer of initial products so that the end consumer can then process this initial product using a current method.

The TP method, wherein the separate preparation of NS and VS is omitted, contains two sub-variants whereby the networking starches are fed jointly (One Feeding, OF) or separately (Split Feeding, SF) to the preparation. The OF variant is preferred if both NS and VS are present as powder or granules and thus can be mixed in a suitable mixing ratio beforehand. The SF variant is preferable if it is difficult or impossible to add NS and VS in a suitable mixing ratio. The Split Feeding variant additionally has the advantage that it is possible to supply VS and NS to the preparation spatially and/or temporally staggered. Thus, for example, it is advantageous to supply the NS to the process when the VS is at least already partially plasticized. By means of the SF variant it is also possible to supply NS and VS with different softener contents, especially with different water contents. In this situation it is advantageous if the NS is added in the swollen state (for example, as suspension or paste), which makes it easier to realise the potential of the NS to form networks. A further possibility of the SF variant involves mixing the NS in an at least partially plasticized state with the VS. In this case, high water contents, high temperatures and high shear forces are advantageously used during the plasticization of NS. This takes place, for example, using an extruder i.e., the NS is prepared in a side extruder and added to the VS. The difference from the SCP method is that the NS is not dissolved but plasticized.

The possible variants of the method which are used depending on specific boundary conditions, needs and the type and desired properties of the specific starch network, overall optimally satisfy the requirements of the manufacturer with regard to ease of implementation and also the requirements with regard to the properties of the end product. The process variants developed for this purpose are summarised in an overview in FIG. 1.

### Initial Products (VP)

With regard to the starch components, the initial product (VP) can have VS or NS or VS and NS, with regard to NS all groups of NS specified further below under NS can be considered. The initial product is obtained in a storable and transportable form, for example, as powder, spray-dried powder, granules, pellets and the like, as a result of which the advantageous possibilities of the DP method are obtained.

The states or variants of the initial product specified subsequently are adjusted both via the composition of the networking starch fluid (NSF) and via the parameters of the method.

VVP: the manufacture of the initial product can, on the one hand, be controlled such that the NSF (NSF1) obtained during the preparation already forms a network to an appreciable extent during its conversion to the initial product, wherein a partially cross-linked initial product (VVP) is obtained. This possibility is especially meaningful if the crystallites forming the linking points of the network have a relatively low melting point so that the network can be dissolved again during the conversion to an NSF (NSF2) which takes place subsequently and can then be re-formed under controlled conditions. On the other hand, the VVP which for example has a water content of 7% and a softener content of 25% can be plasticized as a result of the high viscosity with high shear forces, whereby a network which has been formed can be dissolved again. Such relatively easily plasticisable VVP are preferably based on short-chain amyloses (short chain amylose), gelling dextrins, debranched maltodextrins or hydrolysed starches.

IVP: on the other hand, it is frequently advantageous if the initial product has not yet formed any network or has only formed this to a very limited extent which makes it easier to process the initial product, i.e., it is not necessary to re-dissolve a starch network already obtained. In this case, the initial product is designated as an inhibited initial product (IVP). The essential process measures for manufacturing an inhibited initial product are the rapid reduction of the water content of the NSF, preferably with a simultaneous reduction in temperature so that the NSF can be obtained frozen in an amorphous state. Under suitable conditions such an IVP can be manufactured on the basis of any NS.

KVP: in a third type of initial product, a network is specifically adjusted so that the fractions of ordered structures obtained in this situation, which can act as network elements or as potential network elements, can be used in the subsequent processing of the initial product as nuclei for the starch network which is strived for in the end product. Such an initial product is designated as a nuclei-containing initial product (KVP). Instead of these nuclei, foreign nucleating agents can also be used but these are somewhat less effective. A KVP can be manufactured on the basis of any NS, the process parameters are decisive, wherein only minimal network formation takes place and the nuclei-containing NSF obtained is then frozen. During the subsequent plasticization of KVP, the process parameters, especially softener content, temperature and shearing are adjusted so that the nuclei obtained in the KVP are not destroyed. It is sensible to use a KVP if the network formation in the end product is to take place under difficult conditions, i.e., for example, at low water contents, wherein very high network densities can then be achieved and correspondingly high mechanical strengths, even in the swollen state (low swelling capacity). Such networks, especially if they have additionally been subjected to another heat treatment, have astonishing strengths up to several MPa even after storage in water (where TPS swells and then breaks down and finally dissolves).

For the manufacture of initial products of the type IVP and KVP it is important that the initial product is brought as rapidly as possible to a temperature around or below the glass transition temperature T_{g} since the state of the NSF obtained up till then is frozen. It was astonishingly found that around or even slightly above T_{g}, an NSF is inhibited with regard to network formation, is quasi-frozen, which results in interesting possible applications since the NSF can be shaped very well in this state. In practice, however, the temperature is frequently pre-determined by the room temperature and it is not logical to bring an IVP, for example, in a refrigerator van to the end consumer. Since the glass transition temperature T_{g} depends strongly on the water content, with T_{g} increasing rapidly with decreasing water content, instead of the storage temperature, it is thus possible to adjust the water content during storage to a value so that T_{g} lies in the range of the room temperature and thus the state of the NSF remains frozen at this temperature.

Both VVP, IVP and also KVP can contain additives, especially foreign nucleating agents, they can be stored, transported and then processed to form end products based on starch networks at an end consumer. For converting the initial product into an NSF, where this involves a plasticization in most cases, in general additional softener and especially water are supplied. In addition, additives can also be added in this processing step, as well as further starches (VS, NS).

### Important Process Parameters

The following information on the suitable process parameters is to be understood as guide values. Depending on the VS and NS used (or mixtures of VS and NS) and on the type of initial product or the end product, the parameters should be specifically optimised for each formulation and deviations from the guide values can also occur.

The water content of the NSF before any removal of process water which may be carried out, relative to the dry starch and water in wt. %, lies in the range of 15-70, preferably 20-60, more preferably 25-50, most preferably 30-45. The lower values are used when high mass temperatures and high shear rates are used, the higher values when NS with high CLn,na are used, which are present with high degrees of crystallinity. Water contents higher than 70% can also be used for NS which is to be prepared simply if the initial product is to be obtained in the freeze-dried state.

The softener content of softeners other than water in the NSF is primarily pre-determined not by the method but by the use of the end product to be manufactured from the initial product. The softener content relative to the dry starch and softener in wt. % lies in the range of 0-70, preferably 0-55, more preferably 0-45, most preferably 0-40. In contrast to water, the softener content stays approximately constant during the process or decreases only slightly if some of the softener is removed from the process with the process water (using evacuation techniques). The softener content is usually adjusted so that it corresponds to the desired softener content of the end product which is manufactured from the initial product. However, by adding further softener to the VP, it is also possible to adjust the softener content to higher values during the final processing. Lower softener contents during the manufacture of the VP are especially used during the manufacture of IVP and KVP.

The water content of the NSF after any removal of process water which may take place is an important quantity which decisively influences the type of VP, and relative to the starch and water in wt. % this is 0-35, preferably 0-25, more preferably 0-20, most preferably 0-15. Comparatively low water contents are especially important for producing IVP and KVP in order to suppress network formation. Here also significantly higher water contents can be used if the initial product is to be obtained in the freeze-dried state.

In many cases, after the removal of process water the water content is subsequently reduced still further, where the VP becomes dried. At water contents for which network formation is possible, the drying rate is an important parameter which determines the more or less strong or inhibited formation of a network in the VP.

The maximum mass temperature in ° C. during the preparation of the NSF lies approximately in the range of 80-220, preferably 100-180, more preferably 105-170, most preferably 110-160. The importance of the mass temperature has already been discussed.

### Heat Treatment

After manufacturing shaped bodies, a heat treatment or conditioning is advantageous in some cases. Network formation of the NSF is usually triggered by a reduction in the temperature of the NSF as well as by a reduction in the water content. On the one hand, the cooling conditions can be adjusted so that during the cooling process the desired network density is obtained. In certain cases, it is desirable that the network formation is not complete, and this can be achieved by an accelerated cooling so that the NSF is obtained in a frozen-in state (IVP, KVP) before the network formation is completed. Mostly however, a lower network density is adjusted by the fraction of networking starch used. In most cases, network formation which is as complete as possible is desired at least in the end product. However, this is not always ensured with short cooling times and especially with low softener contents, as well as when using VS and NS having high molecular weights. It is then possible to further increase the network formation subsequently by suitable heat treatment. In addition, this is also advantageous to forestall later changes in the starch network, thus to obtain constant product properties. It is advantageous to proceed during manufacture of the end product such that heat treatment is unnecessary or this takes place of its own accord without further influencing but this is not always possible and especially for high-strength starch networks, heat treatment has hitherto been indispensable. The information specified in the following on advantageous heat treatments or conditioning conditions should be understood as approximate guide values and the optimal values for a formulation depend strongly on the formulation and the water and softener content.

The heat treatment temperature in ° C. lies in the range 0-160, preferably 20-140, more preferably 40-120, most preferably 60-110. The high temperatures are used especially when the NS has a high molecular weight (e.g., long chain amylose) and the softener or water content is low (e.g. 20% water, 0% softener), the low temperatures when the NS has a low molecular weight (e.g., short chain amylose) and the softener and water content is high (e.g. a temperature of 20° C. is sufficient for complete formation of the SCA network with 30% glycerol and 18% water during around 24 h whereas when the water content is reduced to 14% at room temperature, virtually no network formation takes place).

The heat treatment time lies in the range of minutes to days and depends strongly on the temperature. For every increase by 10° C. an approximate doubling of the network formation rate is obtained. In most cases, short heat treatment times are preferred which are correspondingly obtained at high temperatures.

In addition, the water content can be kept constant during the heat treatment or it can have a temporally defined profile, increasing or decreasing. In the event of heat treatment at a pre-determined relative air humidity for example, the water content gradually tends to the equilibrium water content, with increasing or decreasing water content, depending on the relative air humidity and the initial water content of the sample. Thus, a desired water content can also be adjusted by means of a heat treatment at a pre-determined relative air humidity.

### Basic Starch (VS)

Any starch or even a flour in any state, both physically and/or chemically modified, can basically be supplied to the process as basic starch, they can be used in native form or they can have been modified by physical methods such as for example by gelatinisation (partially to completely), plasticization or inhibition.

Examples for basic starches or flours are of the following origin: cereals such as maize, rice, wheat, rye, barley, millet, oats, spelt, roots and bulbs such as potato, sweet potato, tapioca (cassava), maranta (arrowroot), pulses and seeds such as beans, peas, mungo and lotus. In addition, starches and flour of other origin can also be considered such as sago or yams, for example. In addition, glycogen can also be used. The basic starches can have been modified by breeding or genetic engineering methods such as for example, waxy maize, waxy rice, waxy potato, high-amylose-containing maize, Indica rice, Japonica rice.

Furthermore, starches or mixtures of such starches which have been modified by the following treatments or combinations of these treatments can also be used as VS:

oxidation (for example, periodate oxidation, chromic acid oxidation, permanganate oxidation, nitrogen dioxide oxidation, hypochlorite oxidation: oxidised starches); esterification (for example, acetylated starches, phosphorylated starches (monoester), starch sulphate, starch xanthate); etherification (for example, hydroxyalkyl starches, especially hydroxypropyl or hydroxyethyl starches, methyl starches, allyl starches, triphenylmethyl starches, carboxymethyl starches, diethylaminoethyl starches); cross-linking (for example, diphosphate starches, diadipate starches); graft reactions; carbamate reactions (starch carbamates).

Starches with partially substituted hydroxyl groups show advantageous film-forming properties for applications, high elongations such as are especially required for manufacturing films. These properties usually increase with the degree of substitution DS and the size of the substituted group. Thus, starches with DS>0.01 are preferred, more preferably >0.05, especially >0.10, most preferably >0.15. However, the sensitivity of these starches to water also increases with the degree of substitution so that TPS based on these softened starches have barely measurable strengths at RF above 50% (with high DS). However, when incorporated in starch networks, good mechanical properties can be obtained even at the highest RF and thus these exceptional film-forming agents can be used correctly. The upper limit of the DS is determined by regulatory provisions for pharmaceutical applications and foodstuff applications. However, modified starches with higher DS are suitable and advantageous for non-edible applications. Examples of substituted starches of particular interest are hydroxypropylated or hydroxyethylated or acetylated or phosphorylated or oxidised root and bulb starches or waxy starches.

Likewise of particular interest with regard to the viscosity are stabilised VS, i.e., chemically cross-linked starches such as, for example, distarch phosphates, distarch adipates or inhibited starches (Novation starches). Particularly preferred are chemically cross-linked and simultaneously substituted starches, wherein higher degrees of substitution are also favourable for film applications here. An advantage of using substituted and simultaneously chemically cross-linked starches is that a wide range of types with different degrees of substitution and cross-linking of these favourable commodity starches are commercially available in food quality. Examples are hydroxypropylated distarch phosphates, hydroxypropylated distarch adipates, acetylated distarch phosphates or acetylated distarch phosphates which are available based on starches of various origin such as maize, wheat, millet, rice, potato, tapioca etc.

Also of interest are dextrins, especially pyrodextrins such as white dextrins, yellow or canary dextrins, modified dextrins, co-dextrins or British gums. These also have good film-forming properties and as a result of their irregular structure and the high degree of branching Qb of typically >0.05, they are partially to almost completely stable with respect to retrogradation and thus have very good long-term stability, i.e., resistant to ageing and despite this can be used for heterocrystallisation, especially when SCA is used as NS. In addition, the use of dextrins has a positive influence on the welding properties since they have good adhesive properties as long as no significant network formation has yet taken place. Dextrins with low to moderate degrees of conversion can be used as VS alone or together with further VS, whereas dextrins with high degrees of conversion are preferably used together with further VS. With regard to the optical properties, white dextrins are preferred.

A next group of interesting starches are hydrolysed starches such as acid-hydrolysed starches or enzymatically hydrolysed starches as well as chemically modified hydrolysed starches. These starches can be used both as VS and as NS.

Finally, of particular interest are basic starches whose amylopectin fraction has an average chain length CL>20, preferably >22, more preferably >24, most preferably >26 since side chains of this magnitude together with NS, especially together with SCA of the same magnitude, can heterocrystallise very well, with high network densities being obtained.

Basic starches are used, for example, in powder form, basically all forms of preparation can be used, for example, spray-dried forms, drum-dried forms, granules, pellets and the like. Mixtures of various basic starches can also be used as basic starch.

### Networking Starch (NS)

Starches containing or consisting of amyloses or amylose-like starches are used as NS. A mixture of various NS types is also designated as NS. The amyloses can be both linear as well as branched and if necessary modified. Examples for NS are amyloses from native starches, especially amyloses obtained by fractionating starches having an amylose content >23%, modified amyloses, especially substituted amyloses or hydrolysed amyloses, synthetic amyloses, cereal starches, pea starches, high-amylose-containing starches, especially having an amylose content >30, preferably >40, more preferably >60, most preferably >90, hydrolysed starches, especially hydrolysed high-amylose-containing starches or sago starches, gelling dextrins, fluidity starches, microcrystalline starches, starches from the field of fat replacers. In addition, NS can also have an intermediate fraction such as are obtained for example in high-amylose starches and can be obtained by fractionation. With regard to their structure and properties, the intermediate fraction lies between amylose and amylopectin.

For amylose it is usual to distinguish between long chain amylose (LCA) with DPn>100 and short chain amylose (SCA) with DPn<100. Networking starches can have LCA and/or SCA.

### Short Chain Amylose (SCA)

Examples of SCA are amylodextrins, linear dextrins, Nägeli dextrins, Lintnerised starches, erythrodextrins or achrodextrins which represent various designations and subgroups of SCA.

SCA can be obtained, for example, by hydrolysis of LCA, LCA-amylopectin mixtures or amylopectin mixtures. For advantageous networks, particularly suitable SCA is obtained for example, by hydrolysis of starches originating from roots and bulbs or from heterowaxy or waxy starches. The hydrolysis can take place chemically such as, for example, acid hydrolysis and/or enzymatically such as for example by means of amylases or combinations of amylases (alpha-amylase, beta-amylase, amyloglucosidase, isoamylase or pullulanase). Amylose-containing starches are obtained by combined acid/enzyme hydrolysis as SCA, wherein the two hydrolyses can take place simultaneously or successively. Depending thereon, different types of SCA can be obtained starting from the same starch. In addition, the characteristics of SCA are also influenced by the state of the native starch during the hydrolysis, for example, by the degree of swelling of the starch grains. Thus, there is a wide range of suitable SCA available. Further types can be obtained by acid/enzyme hydrolysis or enzyme hydrolysis starting from waxy starches, wherein SCA hydrolysates with DPn typically around 22 are obtained, which are particularly suitable. Also of particular interest is SCA formed during the process of preparing the starches to form NSF and finally to form the starch network, for example by pullulanase.

### Long Chain Amylose (LCA)

The amylose contained in native starch is usually LCA with DPn>100. However, the degree of polymerisation DPn of LCA can be reduced for example by acid hydrolysis and/or enzymatic hydrolysis and/or oxidation to values <100, so that correspondingly modified native starches can also have SCA.

Numerous methods for producing SCA, LCA and mixtures of SCA and LCA are described in the prior art. Both types of amylose can be obtained on the one hand in pure form and are contained in various, possibly hydrolysed commercial starches in different fractions.

It is pointed out that in certain cases, especially in SCP and SDP methods, VS and NS can be identical in terms of material since in principle every NS can also be used as a VS. The difference between VS and NS is thus not of a material nature in all cases, rather the terms must also be understood in connection with the method. NS is treated in a manner that optimally releases its potential for forming networks whereas this need not be the case with VS.

The mechanical properties, primarily the E modulus and strength, are significantly positively influenced by the formation of a starch network consisting of NS and VS, this influence being more marked, the higher the network density. The network density is primarily dependent on the type and the fraction of NS, on the specific combination of NS and VS, additionally on the processing conditions (temperature, softener content, water content, shear rate), the cooling conditions (cooling time) and any heat treatment that may be carried out. Thus, significantly improved mechanical properties can be obtained on the basis of starch networks compared with conventional thermoplastic starch (TPS).

### Activation and Stabilisation of the NS in the SCP and SDP Methods

In order to adjust a defined network, NS and possibly VS is activated and especially stabilized before or during the mixing with VS. As a result of the activation, it is achieved that the amylose contained in the NS is present in the amorphous state so that after the mixing with VS recombination can take place, which results in a network. As a result of the stabilisation it is possible to influence the beginning of network formation and the type of network.

The higher the water content and the larger the shear forces during the plasticization or dissolution process, the lower the necessary temperatures. Of particular importance is an activation associated with stabilisation of the NS. The stabilisation is achieved by overheating the amylose to temperatures above the melting or dissolution process. In addition, foreign nucleating agents and/or methods can be used to produce suitable nuclei by means of undercooling the activated NS. For detailed information relating to activation, stabilisation, nuclei formation, undercooling and foreign nucleating agents reference is made to the Patent Applications WO 03/035026 A2 and WO 03/035044 A2.

By means of the stabilisation, the temperature of the recombination of amylose to the desired network can be adjusted to low temperatures. The higher the stabilisation or overheating temperature, the lower the temperature at which recombination or network formation takes place for the same water and softener content.

### Softener

With regard to softeners (WM) there is a wide range of known starch softeners to choose from which have been described on many occasions in the prior art (see for example WO 03/035026 A2 or WO 03/035044 A2). Mention may be made by name here of the polyols glycerol, erythritol, xylitol, sorbitol, mannitol, galactitol, tagatose, lactitol, maltitol, maltulose, isomalt. These and further softeners can respectively be used alone or in various mixtures. It was found that for starch networks especially suitable softeners have melting points <100° C., preferably <70° C., more preferably <50° C., most preferably <30° C. Water is by far the most important softener, around 2.5 times more effective than glycerol. Here however, water is mostly not designated as a softener in order to distinguish water from the other softeners.

Softeners are supplied to the starches at the beginning of the process, they are required to plasticize the starches and convert into a fluid. Softeners can also be supplied to the process at a later point or be partially removed from the process. The softener water plays a particular role in this respect, on the one hand because water is the cheapest and most efficient softener and on the other hand because the water content can easily be varied during the process. Thus, for example, in the TCP and TDP methods high water contents are initially used to plasticize VS and especially NS whereby the potential for networking of NS is released at simultaneously high temperatures. Thereafter, the water content is reduced again, for example, by evacuation techniques. A further variation of the water content is possible, if necessary during the conditioning of initial or end products. Thus, the softener water provides large scope in the process whereas other softeners can only be removed from the process again with difficulty.

### Types of Sugars

Types of sugars such as glucose, galactose, fructose, sucrose, maltose, trehalose, lactose, lactulose, raffiniose, glucose syrup, high maltose corn syrup, high fructose corn syrup, hydrogenised starch hydrolysates are used on the one hand if water solubility or breakdown of the network in aqueous media is desired or in order to improve the barrier properties. They also partly influence the sorption behaviour.

### Admixtures and Blends

With regard to admixtures such as foreign nucleating agents, additives, fillers, propellants, with regard to an arrangement of hydrocolloids which can be mixed with an NSF and with regard to the synthetic polymers with which starch networks can be processed to form blends which can be obtained as an initial product or which can be produced with an initial product, reference is made to the Patent Application WO 03/035026 A2 and only the most interesting synthetic polymers may be mentioned here by name: polyvinyl alcohols, both partly and completely hydrolysed types, polyethylene glycols, polyethylene oxide, polyvinyl pyrrolidone, polycaprolactone.

### EXAMPLES

### Properties

The following discussion on FIG. 1 relates to a specific formulation and various methods for producing starch networks in an end and initial product and shows the difference between starch networks and thermoplastic starch. However, the situation is basically valid for various formulations for starch networks and TPS wherein through the choice of VS, NS, softener and the softener fraction as well as by using further substances, the characteristic of the E modulus curves from FIG. 1 remains the same. The afore-mentioned parameters primarily bring about a shift of the curves along the RF axis and/or along the E modulus axis and/or a stretching or compression along these axes. Furthermore it is possible that the region of the quasi-plateau of the E modulus is more or less prominent, or is completely missing.

FIG. 1 shows the behaviour of the E modulus as a function of the air humidity for a formulation based on hydroxypropylated starch with moderate DS containing 10% NS and 32% softener for various methods of manufacture as well as for a comparable formulation but containing no NS, that is for TPS. The E modulus is a material property which on the one hand is of major importance for the application and on the other hand, the E modulus, especially at moderate to high RF, is significantly influenced by a starch network, wherein there is a proportionality between the extent of the network or the network density and the E modulus. The E modulus and its behaviour with RF is thus a suitable parameter for illustrating the network formation.

The comparison of the E modulus for the SCP method (SC77) with TPS (SC83) clearly shows the difference between a thermoplastic starch based on the same starch and starch networks. At low RF around 23% the difference is relatively small, the E modulus of SC77 being somewhat increased as a result of the contribution of the network. With increasing air humidity, water is absorbed from the atmosphere which exerts a strongly softening effect. This has the result that the E modulus of TPS in a semi-logarithmic plot decreases almost linearly with increasing RF, very rapidly becomes soft and takes on the character of a highly viscous fluid. The reduction of the E modulus is especially prominent for substituted starches which exhibit exceptionally good film forming capacity, whilst non-substituted starches exhibit a flatter profile of the E modulus and can still have measurable E moduli at high RF but they show only low breaking elongation and marked brittleness at low RF. Compared to TPS(SC83), the E modulus lies at significantly higher values as a result of the developed network of SC77 and astonishingly high values are still obtained at high RF. In the case of SC77 the E modulus could be stabilised at moderate RF even at around 10 MPA over a wide range, since the contribution of the network to the E modulus depends comparatively little on the RF or the water content. This has the consequence that even at 75% RF, SC77 still has an E modulus which is comparable with the E modulus of similar TPS at around 40% RF. The advantage of starch networks compared to TPS is thus evident.

Whereas SC77 was produced in the SCP process, where NS was first dissolved and then supplied in this form to a starch melt consisting of VS and the sample was obtained directly from the homogenised mixture of VS and NS, i.e., from the networking starch melt (NSF), the E moduli of the experiments SC183 and SC184 show that the same advantageous results, i.e., fully developed starch networks, can also be obtained using a simplified method, wherein VS and NS were prepared together without preliminarily dissolving NS. For this purpose corresponding process parameters are important, especially a high water content (process water which is at least partly removed from the process again), high temperatures and high shear forces whereby the partly crystalline NS can be distributed in a molecularly disperse fashion in the VS melt. In the case of SC183, according to a first sub-variant of the TCP method, the NS was supplied in powder form to a VS melt containing a softener and process water by means of Split Feeding whereas in the case of SC184, both NS and VS were added together in powder form by means of Together Feeding, mixed with softener and process water and then plasticized.

In experiment SC184 L2 less process water was used compared with SC184, as a result of which only some of the NS could be dispersed in the VS melt in a molecularly disperse fashion and some of the NS was obtained still in crystalline or partially crystalline form as dispersed particles in the starch melt. Thus, an NSF having a reduced content of molecularly disperse basic and therefore networking NS molecules was obtained and therefore a reduced network density in the end product, i.e. the potential of the NS was only partly realised in experiment SC184 L2. In experiment SC184 L1 the same quantity of process water was used as in SC184 but the experiment was carried out at lower speeds as a result of which the shear forces were lower and lower mass temperatures were obtained. The result is comparable as in SC184 L2. Overall, by varying the process parameters, i.e., by continuously reducing the fraction of process water and/or the temperature and/or the shear forces compared with SC77, the entire range of E moduli curves between SC77 and TPS (SC83) can be obtained with which all states between a fully developed starch network and no starch network can be adjusted. Fully developed networks are naturally preferable for the end product but the products of SC184 L2 and L1 can be useful as initial products, where the dispersed NS particles can be used in a next processing step as nuclei for an optimal network formation in the end product, especially nuclei consisting of SCA in the initial product for network formation under difficult conditions in the end product (with low water content) or when LCA is used simultaneously, which is added as solution and is distributed in a molecularly disperse fashion in the initial product but is quasi-frozen in this state (inhibited with regard to network formation) and cannot form any network.

E-moduli curves between the curves of SC77 and TPS (SC83) can also be obtained if the NS is dispersed in the VS melt in a completely molecularly disperse fashion, thus an NSF is present in an optimal state for subsequent network formation, but the process is then carried out, for example, by removing process water and/or by reducing the temperature so that the formation of a starch network is partially to completely suppressed and the state of the NSF is frozen-in. Thus, an initial product partially to completely inhibited with regard to network formation (IVP) can be obtained which can easily be plasticized again at a later point in time and yields a completely developed network in the end product under suitable conditions.

Thus, the various types of initial products can also be characterised by means of FIG. 1. The E-modulus behaviour of SC77 corresponds to a completely cross-linked initial product (VVP), the range of E-modulus curves between SC77 as far as SC83 corresponds to increasingly less cross-linked initial products (if the NS is distributed in a completely molecularly disperse fashion in the VS melt) or increasingly inhibited initial products whereas the E-modulus behaviour of SC83 corresponds to a completely inhibited initial product (if the NS contained in the NSF in a molecularly dispersed fashion is frozen-in in this state, there is almost no difference from TPS regardless of the formulation, the difference only arises with the onset of network formation). On the other hand, the range of E-modulus curves between SC77 as far as SC83 when the NS is only present in a partially molecularly disperse fashion in the VS melt, also corresponds to nuclei-containing initial products (KVP).

FIG. 2 shows the E-modulus as a function of the RF for end products manufactured by the SDP method with varying fractions of NS. In the SDP method the NS was prepared separately (Split), supplied to a VS melt in the extruder and an NSF was obtained therefrom, which was shaped by means of a flat-slitted nozzle to give a film 0.5 mm thick and dried in the air flow in the atmosphere at 25% air humidity and thus obtained as an initial product. In this case, SC77 E was obtained as a partially cross-linked initial product (VVP) (having an E-modulus profile approximately corresponding to the sample SC184 L1 from FIG. 1) since it was possible for cross-linking to take place during the drying time before reaching the frozen-in state (about 1 h). In the case of SC173 E and SC172 E an almost completely inhibited initial product (IVP) was obtained (with an E modulus approximately corresponding to the TPS(SC83)) since the concentration of NS was too low to form a network within the drying time. The dried film was then reduced to a grain size of about 1 mm and re-plasticized using a Brabender kneader whilst supplying process water (30%) at around 110° C., and after reducing the process water to 20% by means of atmospheric degassing, formed into an end product in the form of a film 0.5 mm thick using a plate press.

The profile of the E modulus of SC77 E (SDP method) has somewhat higher values than that for SC77 (SCP method, FIG. 1). The difference arises because SC77 was produced using a Brabender kneader and the initial product of SC77 E was produced using an extruder, whereby better homogenisation of the NSF could be obtained, that is a more optimal molecularly disperse distribution of the NS is obtained.

The samples SC172 E and SC 173 E containing only 2 or 3% NS surprisingly show an E-modulus profile which lies significantly above the E modulus of TPS(SC83), i.e., the advantages of the network are already obtained with minimal fractions of NS.

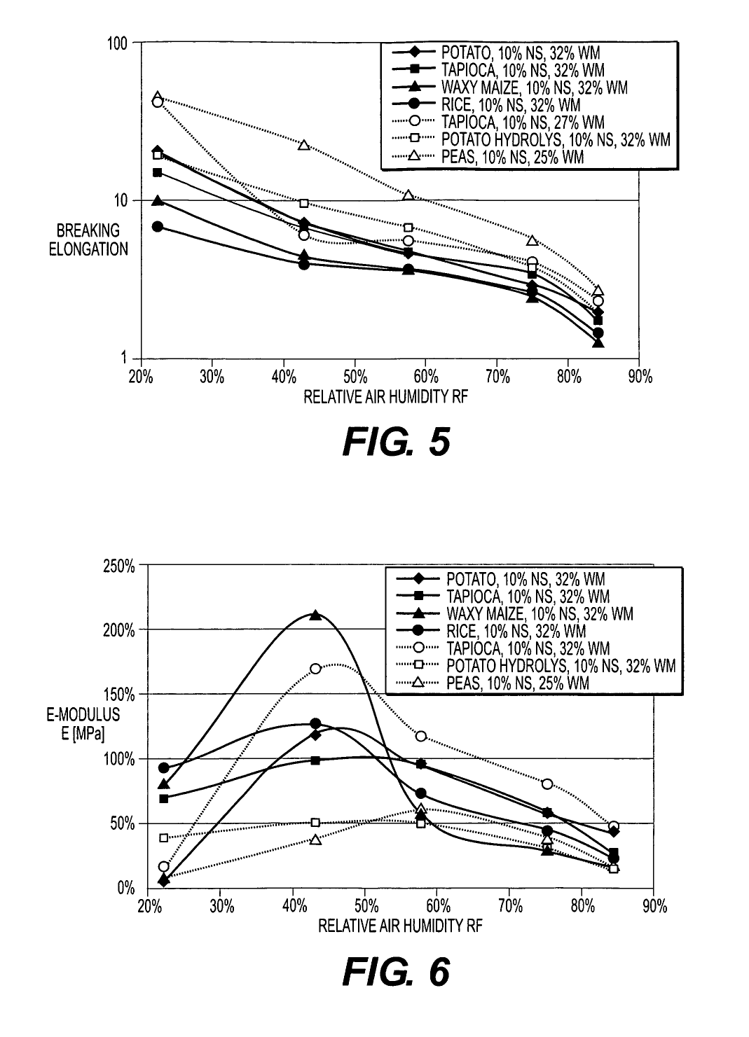

FIG. 3 shows the E-modulus curves for various network starches based on hydroxypropylated starch having moderate DS as VS and a softener content of 32% with 10% NS manufactured by the TDP method, where a spectrum of various NS types was studied:

| | | H₂O | Tₘ |
|---|---|---|---|
| No. | NS | [%] | [° C.] |
| SC77 E | Short chain amylose, DPn = | 30 | 135 |
| | about 2 0 | | |
| SC149 F | Long chain amylose, DPn = | 33 | 135 |
| | about 300 | | |
| SC136 F | Long chain amylose, DPn = | 36 | 177 |
| | about 700 | | |
| SC147 F | Tapioca dextrin | 28 | 115 |
| WS118 F | Maize, acid enzyme | 35 | 150 |
| | hydrolysed | | |
| WS115 F | Potato, hydrolysed | 28 | 110 |
| | oxidised | | |
| SC200 F | Potato dextrin | 28 | 110 |
| WS119 F | Maize, acid hydrolysed | 34 | 145 |

In the TDP method the NS was supplied in powder form to a VS plasticized by means of an extruder (Split Feeding) and homogenised with this to form an NSF with a molecularly dispersed distribution of NS, wherein the water contents (H₂O) and the maximum mass temperatures Tₘ were adapted for the respective NS. The softener content during the extrusion was 20%. The NSF was finally extruded as strand via round nozzles having a diameter of 1 mm. At higher mass temperatures the strand was severely expanded whereby the water content reduces very rapidly and the amorphous state of the NSF is thus immediately frozen-in, and the intermediate product was thus obtained without network (IVP). At mass temperatures of 115 to 110° C., the expansion was significantly lower and the water content was reduced accordingly (to around 24%). The strands obtained were then dried to a water content of around 10% in the air flow at 20% RF. During the drying time partial network formation could take place in the case of SC147 F, WS118 F and SC200 F so that the corresponding initial products were obtained in the form of VVP although the network formation was relatively small since the corresponding NS with 24% water content and 20% softener form networks relatively slowly. The dry strands were reduced to a grain size of about 1 mm and then further processed in a Brabender kneader to form the end product, wherein, in addition to the process water, additional softener was also added so that the softener content was increased to 32%. After processing to give the end product, the water content was in the range of 20-25% so that network formation could proceed rapidly.

As shown in FIG. 3, in all the NS studied a significant effect was obtained compared with TPS although there are individual differences between the various NS, especially with regard to the formation of a quasi-plateau and the E modulus at 75 and 84% RF.

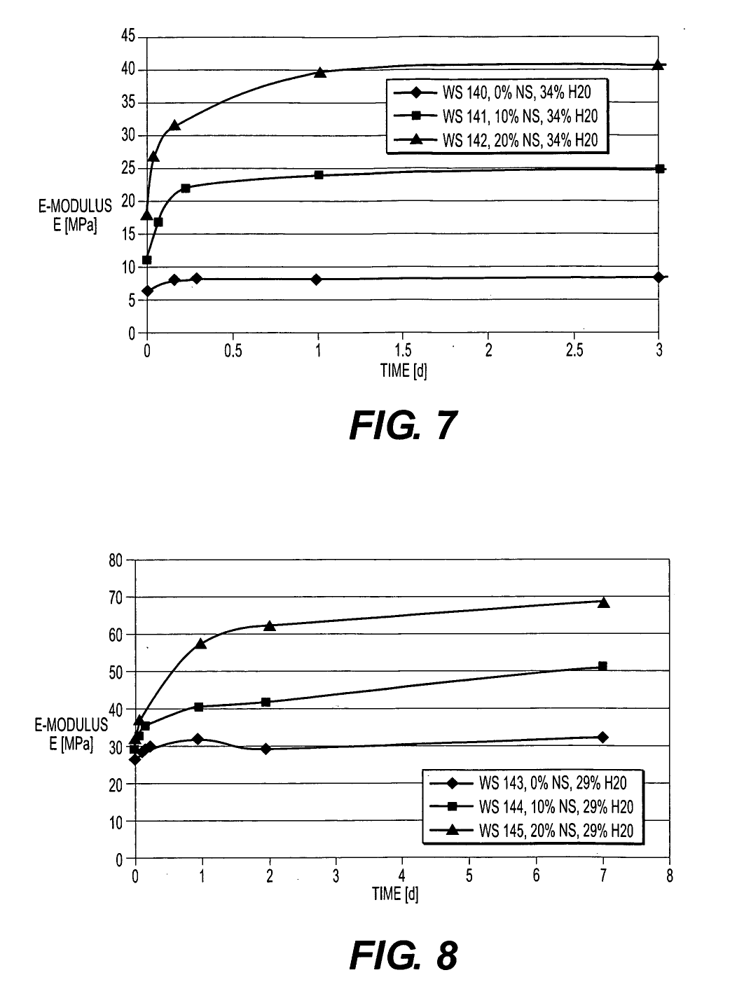

FIG. 4 shows the E-modulus curves for starch networks with respectively 10% NS (short chain amylose with DPn=about 20) based on various VS (not modified, except for the hydrolysed potato starch) manufactured in the TCP method by means of Split Feeding, where the NS was supplied as a suspension in water (but not dissolved) at 80° C. to the plasticized VS in the Brabender kneader and with around 35% water, at mass temperatures of around 115° C. and a speed of 180 rpm, a molecularly disperse mixture of VS and NS was produced. As a result of atmospheric degassing after a kneading time of around 15 min a final water content of about 25% was obtained, whereafter the NSF was formed into a film in a plate press (95° C.). FIG. 4 shows that the starch networks obtained can be produced on the basis of various VS. The E moduli of the VS under study without NS (i.e., the similar TPS, not shown) all lie at lower values, being around 2-8 times lower. In addition, the TPS samples are sticky at RF>40-50%, the stickiness increasing with increasing RF. The corresponding RF networks are not sticky over the entire RF range, for which the reasons lie in the adjusted starch networks.

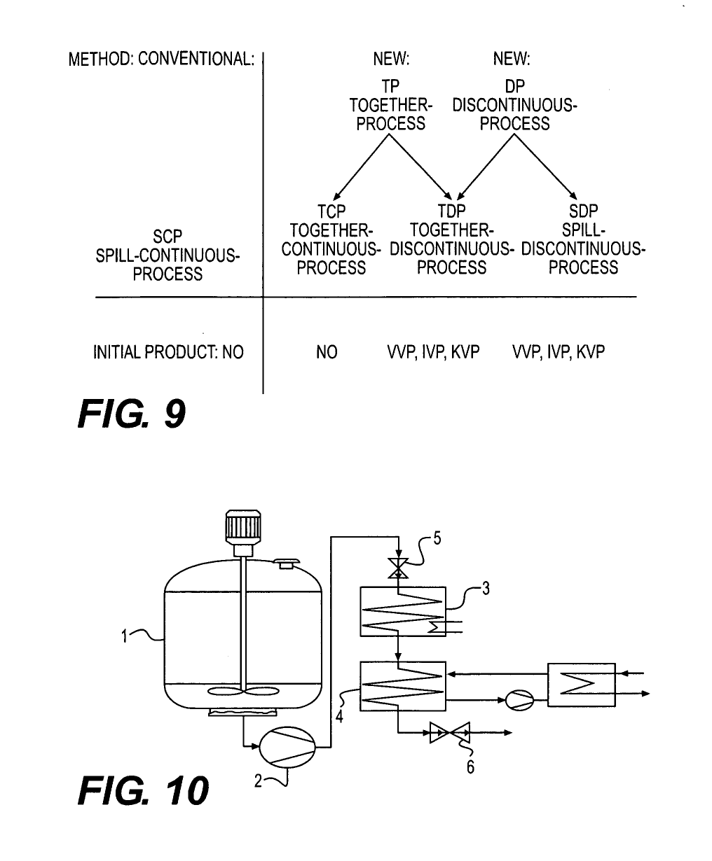

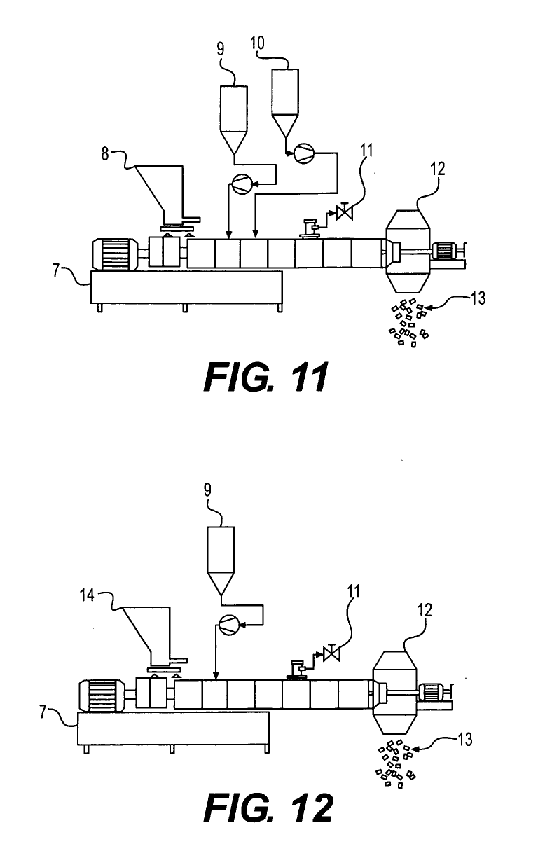

FIG. 5 gives the strengths and FIG. 6 gives the breaking elongations of the same samples as a function of the RF. The strengths of the similar TPS are 2-3 times lower whereas the breaking elongations of the similar starches are comparable or somewhat higher.

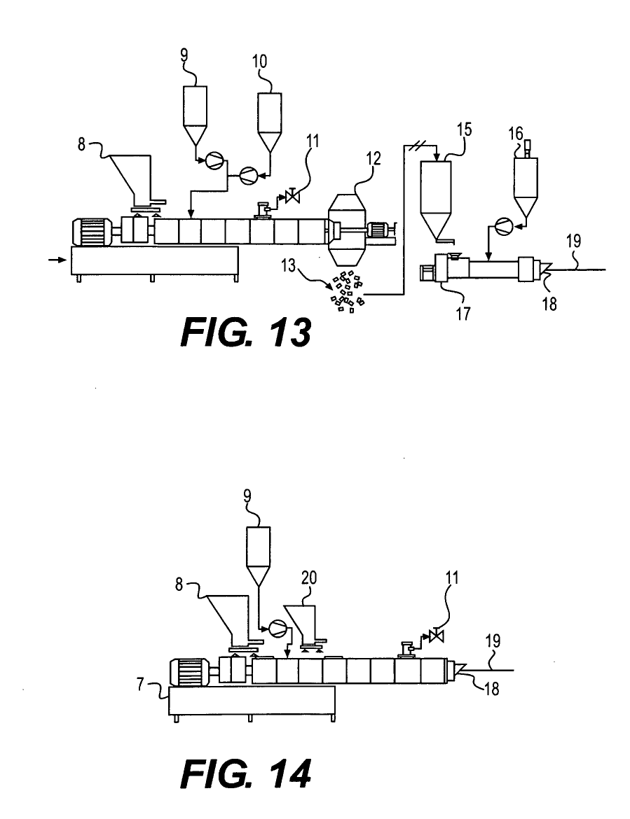

FIG. 7 shows the E moduli of samples based on pre-gelatinised potato starch (not modified) containing 0, and 20% NS (short chain amylose with DPn=about 20), 0% softener and 34% water after manufacture as a function of time (storage at room temperature with constant water content). The manufacture was comparable to the sample in FIGS. 4-6 although the water content of the mixture after adding the NS suspension was 43%. The TPS sample WS140, which was produced similarly to the samples WS141 and WS142, initially shows a slight increase in the E modulus as a function of time, whereafter the E modulus remains constant. In contrast thereto, the E modulus of WS141 increases to more than twice the initial value in the first few hours after which a slower increase in the E modulus can be observed. This growth of the E modulus directly reflects the gradual development of the starch network. Since WS142 with 20% NS has a higher NS content, the growth of the E modulus with time on the one hand takes place more rapidly and on the other hand significantly higher values are obtained. After about 24 h, the network is almost completely developed in WS142 and WS142. If this network formation is suppressed in the course of this 24 h by reducing the water content (where the glass transition temperature rises to temperatures above room temperature), the state attained becomes frozen in and initial products are obtained, wherein after 24 h a completely cross-linked initial product is present, before that partially cross-linked initial products which have a decreasing degree of cross-linking with decreasing storage time. Even when the storage time is 0 h, i.e., after cooling the samples in the press, partial cross-linking of around 50% has already occurred in both samples having NS (which has taken place during the pressing and cooling process, at higher temperature the cross-linking takes place more quickly, the rate of network formation being doubled at 10° C. above room temperature, quadrupled at 20° above etc.). Thus, in order to obtain the samples WS141 and WS142 as completely inhibited initial products, a rapid cooling of the NSF and a rapid reduction in the water content is required, which can be achieved for example by extrusion at a mass temperature >>100° C. and expansion.

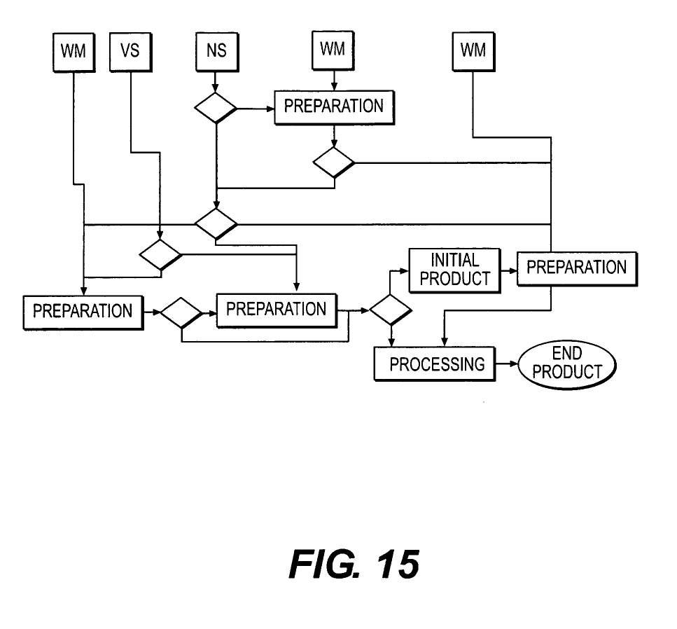

In FIG. 8 the development of the networks with the storage time is illustrated using the corresponding E moduli for comparable samples, where the water content during storage was adjusted to 29%. A comparison of FIG. 7 with FIG. 8 shows that the E-moduli of the samples having NS with the lower water content of 29% increases over a significantly longer time for which the reason is that the rate of formation is significantly reduced as a result of the lower water content. During the pressing process no significant network formation has taken place since the samples WS143, WS144 and WS145 all have the same E modulus (the E modulus of similar TPS) at time 0 h.

The following example shows that starch networks have an astonishing potential with regard to mechanical properties: a sample in the form of film 0.5 mm thick based on a high-amylose-containing (around 60-70% amylose) pre-gelatinised pea starch as VS, containing 35% short chain amylose with a DPn of around 20 as NS, which was produced using the TCP method (Split Feeding) on a Brabender kneader, where mass temperatures of around 120° C. were achieved and most of the process water of initially 50% had been removed by atmospheric degassing, still had a water content of around 18% during shaping in a plate press (apart from water, no other softener was used) and was annealed first in the plate press for 1 h at 120° C., then for 24 h at 95° C. and was finally cooled slowly to room temperature. After swelling in water at room temperature this film showed a strength of 4.7 MPa with which a value almost corresponding to the strength of a pressed polyethylene film (LDPE) was achieved. This is all the more astonishing in that in contrast thereto, TPS typically breaks down in water and thus exhibits no more strength, and frequently even, the strength of TPS is no longer measurable at relative air humidities above 75-85%.

### Method

Illustrations for the method are given in FIGS. 1 to 7.

### Measuring Methods and Conditioning

### Tensile Test

The tensile tests were determined at 22° C. using an Instron 4502 tensile testing machine at a traverse speed of 50 mm/min using standardised tensile samples in accordance with DIN 53504 S3, which have been punched from films around 0.5 mm thick. The measurement results are to be understood as averages of at least 5 individual measurements in each case. The water contents of the tensile samples conditioned at various air humidities were constant during the tensile test to within measurement accuracy. The stress σ was obtained as F/A where F is the force and A is the sample cross-section at ε=0. The elongation in the tensile test in % was obtained as ε=100 (l₁−l₀)/l₀, where l₀ was the extendable length of the sample between the clamps at the beginning of the tensile test and l₁ is the length of the extended sample. The E modulus was obtained as E=σ/ε.

### Conditioning

The samples for the mechanical analyses were conditioned in exsiccators for 7 days at various RF over saturated salt solutions. The exsiccators were fitted with fans whereby the sorption times until equilibrium (7 days) could be significantly shortened compared with storage in a still atmosphere.

### SYMBOLS AND ABBREVIATIONS

RF [%] Relative air humidity: 0%<RF<100%
RT [° C.] Room temperature (22° C.)
T_{g} [° C.] Glass transition temperature
WM [%] Softener content (exclusively water) relative to starch and softener, dsb
W [%] Water content, relative to starch, softener and water
dsb [-] Dry solid base, relative to the dry weight
E [MPa] E modulus (Young's modulus)
σₘ {Mpa] Maximum strength in tensile test breaking strength)
σ_{10%} [MPa] Tensile stress in tensile test at ε=10%
ε_{b} [%] Breaking elongation in tensile test
DP [-] Degree of polymerisation
DPₙ [-] Numerical average of the degree of polymerisation
DP_{w} [-] Weight average of the degree of polymerisation
Q_{b} [-] Degree of branching of macromolecules (number of branched monomer units/number of monomer units)
CL [-] Chain length (number of monomer units)
CLₙ [-] Numerical average of the chain length; linear, i.e., unbranched chain segments
CLn,na [-] Numerical average of the network-active chain length; chain segments which crystallise and can participate in networks, i.e. unbranched and non-substituted and non-sterically hindered chain segments
CL_{w} [-] Weight average of chain length
DS [-] Degree of substitution: 0<DS<3.0
DE [-] Dextrose equivalent: 0<DE<100
VS Basic starch
NS Networking starch
WM Softener, can be a single softener or a mixture of different softeners
SCA Short chain amylose (NS or fraction of NS) with DPn in the range of 10-100; SCA alone cannot form starch networks, only in combination with other starches of higher degree of polymerisation, networks consisting of such mixtures can still be formed with lower softener contents and at low temperatures
LCA Long chain amylose (NS or fraction of NS) with DPn>100, can have LCA1 and/or LCA2
LCA1 LCA with DPn in the range of 100-300; LCA1 can form networks, both alone and in combination with other starches, mixtures of LCA1 and VS can form networks at moderate softener contents and moderate temperatures
LCA2 LCA with DPn>300; LCA2 can form networks, both alone and in combination with other starches. Mixtures of LCA2 and VS can form networks at high softener contents and high temperatures
NSF Networking starch fluid; melt or solution containing a starch or a starch mixture as well as softener; can be obtained under suitable conditions subsequently as starch network. An NSF has at least one VS, as well as at least one NS
VP Initial product obtained from NSF; is obtained as networking starch fluid, is an intermediate product in the DP method
VVP Cross-linked initial product obtained from NSF; has at least partially formed starch network
IVP Inhibited initial product obtained from NSF; has no or only slightly developed network, the formation of a network is suppressed by process measures. An IVP is predominantly to completely amorphous
KVP Initial product containing nuclei, obtained from NSF, has a slightly developed network, whose network elements act as nuclei during the processing of KVP to produce starch networks
SCP Split continuous process: VS and NS are processed separately, mixed to an NSF and the NSF processed directly to the end product
TCP Together Continuous Process: VS and NS are prepared together to give an NSF and the NSF is processed directly to give the end product
SDP Split Discontinuous Process: VS and NS are prepared separately, mixed to give an NSF and the NSF is processed to give a VP
TDP Together Discontinuous Process: VS and NS are prepared together, mixed to give an NSF and this is processed directly to give the end product

### LEGEND

1) Mixing container with NS, water and if necessary WM
2) Liquid pump
3) Heating section
4) Cooling section
5) Check valve
6) Adjustable overpressure valve
7) Extruder
8) Solid metering device for VS
9) Liquid metering device for WM and/or water
10) Liquid metering device with dissolved NS
11) Degassing device
12) Granulating device
13) Initial product (granules)
14) Solid metering device for mixture of VS and NS
15) Solid metering device for initial product
16) Solid metering device for WM and/or water
17) Processing extruder
18) Shaping tool
19) Film
20) Solid metering device for NS

1-15. (canceled)

16. A method for producing starch networks comprising:
preparing a basic starch;
preparing a networking starch;
manufacturing an initial product including the basic starch and the networking starch; and
processing the initial product to yield the starch networks, such that the starch networks are at least partially heterocrystallized.

17. A method according to claim 16, further comprising preparing the basic starch and the networking starch separately, and:
mixing the basic starch and the networking starch to yield a first networking starch fluid;
processing the first networking starch fluid in a first process zone to yield the initial product in a first process zone; and
wherein processing the initial product includes processing the initial product to yield a second networking starch fluid in a second process zone; and
processing the second networking starch fluid to yield the starch networks.

18. A method according to claim 16, further comprising:
supplying the basic starch and networking starch separately, wherein the basic starch is at least partially plasticized, and said manufacturing the initial product includes preparing the basic starch and the networking starch together to yield the initial product, the initial product being a networking starch fluid; and
processing the networking starch fluid directly to yield the starch networks.

19. A method according to claim 16, further comprising:
supplying the basic starch and the networking starch together;
preparing the basic starch and the networking starch together, wherein said manufacturing the initial product includes mixing the basic starch and the networking starch to yield the initial product, the initial product being a networking starch fluid; and
processing the networking starch fluid directly to yield the starch networks.

20. The method for producing starch networks according to claim 16, wherein the initial product is obtained from a networking starch fluid and has an at least partially formed starch network, said method further including at least partially re-dissolving the initial product in a second process zone, and reforming the re-dissolved initial product.

21. The method for producing starch networks according to claim 16, wherein the initial product is obtained from a networking starch fluid, the initial product having an at most partially formed network and an almost completely suppressed network formation, and the initial product is obtained in at least a partially amorphous state, the initial product being inhibited.

22. The method for producing starch networks according to claim 16, wherein the step of processing the initial product includes forming the starch networks from nuclei corresponding to the network regions or elements.

23. The method for producing starch networks according to claim 16, wherein the starch networks include starch macromolecules, said starch macromolecules being dispersed within said networking starch.

24. The method for producing starch networks according to claim 16, wherein the initial product and the starch network include a starch having a network-active chain length, the network-active chain length being in a range of 7 to 300, and the initial product and the starch network include the basic starch and the networking starch, wherein a proportion of a weight of the networking starch relative to a combined weight of the networking starch and the basic starch is in a range of 1 wt. % dsb to 90 wt. % dsb.

25. The method for producing starch networks according to claim 24, wherein the starch network includes a further starch with a degree of branching greater than 0.01.

26. The method for producing starch networks according to claim 16, wherein the initial product and the starch network include a starch having a network-active chain length, the network-active chain length being in a range of 18 to 28, and the initial product and the starch network include the basic starch and the networking starch, wherein a proportion of a weight of the networking starch relative to a combined weight of the networking starch and the basic starch is in a range of 3 wt. % dsb to 15 wt. % dsb.

27. The method for producing starch networks according to claim 26, wherein the starch network includes a further starch with a degree of branching greater than 0.15.

28. The method for producing starch networks according to claim 16, wherein the initial product and the starch networks include amylose, an amount of said amylose in the initial product and the starch networks being in a range of 1 wt. % dsb to 70 wt. % dsb, said amylose being a short chain amylose, a long chain amylose, or a mixture of said short chain amylose and said long chain amylose, wherein the initial product further includes amylopectin, and wherein when said amylose includes said short chain amylose, a proportion of a weight of the short chain amylose to a combined weight of the amylopectin and the short chain amylose is in a range of 1 wt. % dsb to 35 wt. % dsb, and when said amylose includes said long chain amylose, a proportion of a weight of the long chain amylose to a combined weight of the amylopectin and the long chain amylose is in a range of 1 wt. % dsb to 70 wt. % dsb.

29. The method for producing starch networks according to claim 16, wherein the initial product and the starch networks include amylose, an amount of said amylose in the initial product and the starch networks being in a range of 3 wt. % dsb to 30 wt. % dsb, said amylose being a short chain amylose, a long chain amylose, or a mixture of said short chain amylose and said long chain amylose, wherein the initial product further includes amylopectin and wherein when said amylose includes said short chain amylose, a proportion of a weight of the short chain amylose to a combined weight of the amylopectin and the short chain amylose is in a range of 4 wt. % dsb to 14 wt. % dsb, and when said amylose includes said long chain amylose, a proportion of a weight of the long chain amylose to a combined weight of the amylopectin and the long chain amylose is in a range of 5 wt. % dsb to 30 wt. % dsb.

30. The method for producing starch networks according to claim 28, wherein the short chain amylose has a degree of polymerization in a range of 5 to 70, and the long chain amylose has a degree of polymerization in a range of 100 to 3000.

31. The method for producing starch networks according to claim 28, wherein the short chain amylose has a degree of polymerization in a range of 9 to 27, and the long chain amylose has a degree of polymerization in a range of 100 to 300.

32. The method for producing starch networks according to claim 16, wherein the initial product and the starch networks include a material selected from the group consisting of: dextrin, maltodextrine, linear dextrine, amylodextrin, Nägeli dextrine, at least partially debranched or hydrolysed starch, dextrin, or maltodextrin, wherein the at least partially debranched or hydrolysed starch, dextrin or maltodextrin are formed during the manufacture of the initial product; and the initial product and the starch networks include at least one further additive.

33. The method for producing starch networks according to claim 16, wherein the initial product is a networking starch fluid and said networking starch fluid includes a softener, said method further including removing water from the networking starch fluid to form a dry starch, wherein a proportion of a weight of the water in the networking starch fluid before the removal of the water to a combined weight of the dry starch and the water is in a range of 15 wt. % dsb to 70 wt. % dsb; and a proportion of a weight of the softener other than the water of the networking starch fluid to a combined weight of the dry starch and the softener is in a range of 0 wt. % dsb to 70 wt. % dsb.

34. The method for producing starch networks according to claim 33, wherein after any removal of process water, a proportion of a weight of the water to a combined weight of the starch of the networking starch fluid and the water is in a range of 0 wt. % dsb to 35 wt. % dsb; and the maximum temperature during the preparation of the networking starch fluids is in a range of 70° C. to 220° C.

35. The method for producing starch networks according to claim 16, wherein the initial product is a networking starch fluid and said networking starch fluid includes a softener, said method further including removing water from the networking starch fluid to form a dry starch, wherein a proportion of a weight of the water in the networking starch fluid before the removal of the water to a combined weight of the dry starch and the water is in a range of 30 wt. % dsb to 45 wt. % dsb; and a proportion of a weight of the softener content other than the water of the networking starch fluid to a combined weight of the dry starch and the softener is in a range of 0 wt. % dsb to 40 wt. % dsb.

36. The method for producing starch networks according to claim 35, wherein a proportion of a weight of the water to a combined weight of the starch of the networking starch fluid and the water is in a range of 0 wt. % dsb to 15 wt. % dsb; and the maximum temperature during the preparation of the networking starch fluids is in a range of 85° C. to 155° C.

37. The method for producing starch networks according to claim 16, further comprising subjecting the starch networks to a heat treatment with a temperature in a range of 0° C. to 160° C. for a period having a duration of minutes to days.

38. The method for producing starch networks according to claim 16, further comprising subjecting the starch networks to a heat treatment with a temperature in a range of 60° C. to 110° C.

39. A method for producing starch networks from a networking starch fluid, containing a basic starch and a networking starch, comprising:
preparing the basic starch and the networking starch together; and
processing the networking starch fluid directly to yield the starch networks, the starch networks being at least partially formed by heterocrystallization.

40. The method for producing starch networks according to claim 39, further comprising:
supplying the basic starch and the networking starch separately, where the networking starch is supplied to the at least partially plasticized basic starch; and
preparing the basic starch and the networking starch together to yield an initial product, said initial product being the networking starch fluid.

41. The method for producing starch networks according to claim 39, further comprising:
supplying the basic starch and the networking starch together; and
mixing the basic starch and the networking starch to yield the initial product, said initial product being the networking starch fluid.

42. The method for producing starch networks according to claim 39, further comprising:
supplying the basic starch and the networking starch together; and
preparing the basic starch and the networking starch together to yield an initial product, said initial product being a networking starch fluid.

43. The method for producing starch networks according to claim 39, wherein the basic starch is an at least partially plasticized basic starch, the method further comprising:
supplying the basic starch and the networking starch separately, whereby the networking starch is supplied to the at least partially plasticized basic starch; and
preparing the basic starch and the networking starch together to yield an initial product, said initial product being the networking starch fluid.

44. The method for producing starch networks according to claim 39, wherein an initial product is obtained from the networking starch fluid, the initial product having an at least partially formed starch network, said method further including at least partially re-dissolving the initial product in a second process zone and reforming the re-dissolved initial product.

45. The method for producing starch networks according to claim 39, wherein an initial product is obtained from the networking starch fluid, the initial product having an at most partially formed network and an almost completely suppressed network formation, and the initial product is obtained in at least a partially amorphous state, the initial product being inhibited.

46. The method for producing starch networks according to claim 39, wherein an initial product is obtained from the networking starch fluid, wherein obtaining the initial product includes forming the starch networks from nuclei corresponding to the network regions or elements.

47. The method for producing starch networks according to claim 39, wherein the starch networks include a networking starch and starch macromolecules, said starch macromolecules being dispersed within said networking starch.

48. The method for producing starch networks according to claim 39, wherein an initial product is obtained from the networking starch fluid, wherein the initial product and the starch network include a starch with a network-active chain length in a range of 7 to 300, and the initial product and the starch network include the basic starch and the networking starch, wherein a proportion of a weight of the networking starch to a combined weight of the networking starch and the basic starch is in a range of 1 wt. % dsb to 90 wt. % dsb.

49. The method for producing starch networks according to claim 48, wherein the starch network includes a further starch with a degree of branching greater than 0.01.

50. The method for producing starch networks according to claim 39, wherein an initial product is obtained from the networking starch fluid, wherein the initial product and the starch networks include a starch with a network-active chain length in a range of 18 to 28, and the initial product and the starch network include the basic starch and the networking starch, wherein a proportion of a weight of the networking starch to a combined weight of the networking starch and the basic starch is in a range of 3 wt. % dsb to 15 wt. % dsb.

51. The method for producing starch networks according to claim 50, wherein the starch network includes a further starch with a degree of branching greater than 0.15.

52. The method for producing starch networks according to claim 39, wherein an initial product is obtained from the networking starch fluid, wherein the initial product and the starch networks include amylose, an amount of said amylose in the initial product and the starch networks being in a range of 1 wt. % dsb to 70 wt. % dsb, said amylose being a short chain amylose, a long chain amylose, or a mixture of said short chain amylose and said long chain amylose, wherein the initial product further includes amylopectin, and wherein a proportion of a weight of the short chain amylose to a combined weight of the amylopectin and the short chain amylose is in a range of 1 wt. % dsb to 35 wt. % dsb, and a proportion of a weight of the long chain amylose to a combined weight of the amylopectin and the long chain amylose is in a range of 1 wt. % dsb to 70 wt. % dsb.

53. The method for producing starch networks according to claim 39, wherein an initial product is obtained from the networking starch fluid, wherein the initial product and the starch networks include amylose, an amount of said amylose in the initial product and the starch networks being in a range of 3 wt. % dsb to 30 wt. % dsb, said amylose being a short chain amylose, a long chain amylose, or a mixture of said short chain amylose and said long chain amylose, wherein the initial product further includes amylopectin, and wherein a proportion of a weight of the short chain amylose to a combined weight of the amylopectin and the short chain amylose is in a range of 4 wt. % dsb to 14 wt. % dsb, and a proportion of a weight of the long chain amylose to a combined weight of the amylopectin and the long chain amylose is in a range of 5 wt. % dsb to 30 wt. % dsb.

54. The method for producing starch networks according to claim 39, wherein the short chain amylose has a degree of polymerization in a range of 5 to 70, and the long chain amylose has a degree of polymerization in a range of 100 to 3000.

55. The method for producing starch networks according to claim 39, wherein the short chain amylose has a degree of polymerization in a range of 9 to 27, and the long chain amylose has a degree of polymerization in a range of 100 to 300.

56. The method for producing starch networks according to claim 39, wherein the initial product and the starch networks include a material selected from the group consisting of: dextrin, maltodextrine, linear dextrine, amylodextrin, Nägeli dextrine, at least partially debranched or hydrolysed starch, dextrin, or maltodextrin, wherein the at least partially debranched or hydrolysed starch, dextrin or maltodextrin are formed during the manufacture of the initial product; and the initial product and the starch networks include at least one further additive.

57. The method for producing starch networks according to claim 39, wherein the initial product is a networking starch fluid and said networking starch fluid includes a softener, said method further including removing water from the networking starch fluid to form a dry starch, wherein a proportion of a weight of the water in the networking starch fluid before the removal of the water to a combined weight of the dry starch and the water is in a range of 15 wt. % dsb to 70 wt. % dsb; and a proportion of a weight of the softener other than the water of the networking starch fluid to a combined weight of the dry starch and the softener is in a range of 0 wt. % dsb to 70 wt. % dsb.

58. The method for producing starch networks according to claim 57, wherein after any removal of process water, a proportion of a weight of the water to a combined weight of the starch of the networking starch fluid and the water is in a range of 0 wt. % dsb to 35 wt. % dsb; and the maximum temperature during the preparation of the networking starch fluids is in a range of 70° C. to 220° C.

59. The method for producing starch networks according to claim 39, wherein the initial product is a networking starch fluid and said networking starch fluid includes a softener, said method further including removing water from the networking starch fluid to form a dry starch, wherein a proportion of a weight of the water in the networking starch fluid before the removal of the water to a combined weight of the dry starch and the water is in a range of 30 wt. % dsb to 45 wt. % dsb; and a proportion of a weight of the softener content other than the water of the networking starch fluid to a combined weight of the dry starch and the softener is in a range of 0 wt. % dsb to 40 wt. % dsb.

60. The method for producing starch networks according to claim 59, wherein a proportion of a weight of the water to a combined weight of the starch of the networking starch fluid and the water is in a range of 0 wt. % dsb to 15 wt. % dsb; and
the maximum temperature during the preparation of the networking starch fluids is in a range of 85° C. to 155° C.

61. The method for producing starch networks according to claim 39, further comprising subjecting the starch networks to a heat treatment with a temperature in a range of 0° C. to 160° C. for a period having a duration of minutes to days.

62. The method for producing starch networks according to claim 39, further comprising subjecting the starch networks to a heat treatment with a temperature in a range of 60° C. to 110° C.

63. A starch network having, at a relative air humidity greater than 70%, a modulus of elasticity greater by at least a factor of 2, compared with a similar thermoplastic starch (TPS), said starch network being not sticky over a range of 0% to 100% relative humidity, and being almost completely soluble in water, said starch network having a strength in a range of 0.5 mPa to 5 mPa after swelling in water at 22° C., and said starch network being transparent.

64. A starch network according to claim 63, wherein the modulus of elasticity is greater by at least a factor of 5, said strength of said starch network being in a range of 3 mPa to 5 mPa after swelling in water at 22° C.

65. An initial product, including a softener content greater than 30%, said initial product including a networking starch and a basic starch, said initial product being configured to produce starch networks, which are at least partially formed by heterocrystallization, the initial product being in a storable and transportable form, as one of a film, a foil, a powder, a spray-dried powder, a freeze-dried powder, a granule, and a pellet.

66. The initial product, according to claim 65, wherein the initial product is configured to manufacture a product selected from the group consisting of: soft capsules; hard capsules; rubber-elastic confectionery; first foodstuff containing at least one phase comprising a starch network; second foodstuff having a reduced glycaemic index and increased prebiotic fraction; packaging; a foil, a film; a filament; macro- or micro-fibers; a foam; granules; a powder; micro-particles; shaped parts; injection moldings; extruded parts; profile cast parts; deep-drawn parts; thermal shaped parts.

67. The initial product, according to claim 65, wherein the initial product is configured to be produced in foodstuffs, galenicals, cosmetics, health care, packaging or agriculture, polystyrene foam replacement, foil, bioriented foil, composite foil components, membrane system for nano-, micro- or macro-encapsulation, paper laminate, replacement for cellulose, disposable utility clothing, crockery and cutlery, foodtrays, drinking straws, cups, packaging for foodstuffs, in foamed form as a heat-insulating food container, chewing bones for dogs, shopping bags, rubbish and compost sacks, mulch foil, plant pots, golf tips, and children's toys.
